(19) United States
(12) Patent Application Publication
(10) Pub. No.: US 2024/0023822 A1
(43) Pub. Date: Jan. 25, 2024

(54) DYNAMIC IMAGE ANALYSIS APPARATUS, RECORDING MEDIUM, AND DYNAMIC IMAGE ANALYSIS METHOD

(71) Applicant: Konica Minolta, Inc., Tokyo (JP)

(72) Inventors: Ryoichi WATANABE, Tokyo (JP); Takenori FUKUMOTO, Kamakura-shi (JP); Yuzo YAMASAKI, Fukuoka-shi (JP); Kousei ISHIGAMI, Fukuoka-shi (JP); Takeshi KAMITANI, Fukuoka-shi (JP); Tomoyuki HIDA, Fukuoka-shi (JP)

(21) Appl. No.: 18/355,598

(22) Filed: Jul. 20, 2023

(30) Foreign Application Priority Data
Jul. 20, 2022 (JP) 2022-115217

(51) Int. Cl. A61B 5/026; A61B 5/00; G06V 10/25

(52) U.S. Cl. CPC A61B 5/026; A61B 5/0044; A61B 5/7235; G06V 10/25

(57) ABSTRACT

A dynamic image analysis apparatus comprising: a hardware processor, wherein the hardware processor is configured to, execute processing to acquire a dynamic image of a chest obtained by dynamic imaging with radiation, execute processing to generate information on pulmonary valve regurgitation based on dynamic image information of a site related to at least one of a pulmonary artery and a heart in the dynamic image, and execute processing to output the generated information on pulmonary valve regurgitation.

### CROSS-REFERENCE TO RELATED APPLICATIONS

The present invention claims priority under 35 U.S.C. § 119 to Japanese Application, 2022-115217, filed on Jul. 20, 2022, the entire contents of which being incorporated herein by reference.

### BACKGROUND

### Technical Field

The present invention relates to a dynamic image analysis apparatus, a recording medium, and a dynamic image analysis method.

### Description of the Related Art

Long-term stagnation of blood flow in the lungs, heart, or the like due to pulmonary valve regurgitation causes serious life-threatening complications such as right heart failure.

For this reason, when there is pulmonary valve regurgitation or when regurgitation is suspected, it is preferable to periodically perform follow-up observation to determine the timing of re-operation. Pulmonary valve regurgitation is one of the symptoms that occur, for example, in a case in which the subject suffers from tetralogy of Fallot. Tetralogy of Fallot is a congenital heart disease designated as an intractable disease, and in a case where the heart is affected, a cardiac surgical operation is usually performed. However, there is a high possibility that symptoms of pulmonary valve regurgitation will occur after cardiac surgery is performed. In addition, it is known that pulmonary valve regurgitation also occurs in secondary or idiopathic pulmonary hypertension.

As a main method for observing and diagnosing the presence or absence and the degree of pulmonary valve regurgitation, for example, an echo examination (echocardiography), an MRI (Magnetic Resonance Imaging) examination, or the like is used. The echo examination is an examination for depicting an image of a heart, a blood vessel, or the like by using an ultrasonic wave.

A simple diagnosis can be performed by viewing the color Doppler in addition to the ultrasound image in the echo examination using ultrasound. However, the echo examination using ultrasonic waves needs a technical power of a probe operation. For this reason, while pulmonary valve regurgitation is suspected in the clinical data or the like, the clinical data and the echo index may be dissociated from each other due to poor quality of the transthoracic echo diagram. In this case, an MRI examination is eventually required.

In the MRI examination, in addition to simple diagnosis (diagnosis of the presence or absence of regurgitation), quantitative evaluation (detailed examination such as calculation of a regurgitation rate) of the degree of regurgitation can be performed. However, the MRI examination has a limitation that imaging cannot be performed in a case where metal that does not comply with MRI exists in a body of a patient. Further, in the case of an MRI examination, the imaging time is also relatively long, and it is necessary to stand still in a narrow and loud device during imaging. Therefore, it may be difficult to use it particularly for patients with claustrophobia or mental retardation.

In this point, for example, Japanese Unexamined Patent Publication No. 2020-14562 discloses a method of analyzing blood flows such as a pulmonary blood flow and a cardiac blood flow using dynamic images of chest X-rays which are consecutively captured.

If it is possible to determine the presence or absence of pulmonary valve regurgitation by performing blood flow analysis using an X-ray dynamic image, it can be expected to widely respond to diagnostic needs for pulmonary valve regurgitation by a simple method with little burden on patients.

### SUMMARY

However, as described in Japanese Unexamined Patent Publication No. 2020-014562, the technique of blood flow analysis using an X-ray dynamic image has been used for diagnosis of pulmonary embolism, but its use for diagnosis of pulmonary valve regurgitation has not been studied yet.

The present invention has been made in view of the above points, and an object of the present invention is to provide a dynamic image analysis apparatus, a recording medium, and a dynamic image analysis method capable of supporting diagnosis of pulmonary valve regurgitation by performing image analysis of a dynamic image, which is a relatively simple technique with a small burden on a subject.

To achieve at least one of the abovementioned objects, according to an aspect of the present invention reflecting one aspect of the present invention, a dynamic image analysis apparatus includes: a hardware processor, wherein the hardware processor is configured to, execute processing to acquire a dynamic image of a chest obtained by dynamic imaging with radiation, execute processing to generate information on pulmonary valve regurgitation based on dynamic image information of a site related to at least one of a pulmonary artery and a heart in the dynamic image, and execute processing to output the generated information on pulmonary valve regurgitation.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are no intended as a definition of the limits of the present invention, wherein:

FIG. 1 is a diagram showing an image imaging system including an analysis apparatus according to the present embodiment.

FIG. 2 is a diagram illustrating an example of an X-ray dynamic image of a chest and is an illustrative diagram illustrating a case where target regions are set in a heart (ventricle) and a lung field.

FIG. 3 is a flowchart showing an example of a flow of a dynamic image analysis process in a case where information on pulmonary valve regurgitation is a blood flow waveform.

FIG. 4 is a diagram showing an example of a blood flow waveform of a site related to a pulmonary artery.

FIG. 5 is a diagram showing an example of a blood flow waveform at a site related to a pulmonary artery in a healthy person.

FIG. 6 is a diagram showing an example of a blood flow waveform at a site related to a pulmonary artery in a patient with pulmonary valve regurgitation.

FIG. 7 is a flowchart showing an example of a flow of a dynamic image analysis process in a case where information on pulmonary valve regurgitation is skewness and kurtosis obtained based on a blood flow waveform.

FIG. 8 is an explanatory diagram illustrating skewness.

FIG. 9 is an explanatory diagram illustrating kurtosis.

FIG. 10 is a flowchart showing an example of the flow of a dynamic image analysis process in a case where information on pulmonary valve regurgitation is a predicted regurgitation rate and a determination result based on the predicted regurgitation rate.

FIG. 11 is a diagram showing a graph example of a calculation result when a predicted regurgitant flow rate is calculated by multiple regression analysis.

FIG. 12A is an explanatory diagram for explaining a signal value ratio of a heart and a lung field and illustrates the case of a healthy subject.

FIG. 12B is an explanatory diagram for explaining a signal value ratio between the heart and the lung field and shows a case of a patient having pulmonary valve regurgitation.

FIG. 13 is a graph showing a signal value of a blood flow waveform in a target region set in the heart.

FIG. 14 is a graph showing a signal value of a blood flow waveform in a target region set in a lung field.

FIG. 15 is a flowchart illustrating an example of a flow of a dynamic image analysis process in a case where the information on pulmonary valve regurgitation is a signal value ratio of the heart and the lung field.

FIG. 16 is a graph showing the relationship between the signal value ratio of the heart and the lung field and the regurgitant rate of pulmonary valve regurgitation.

FIG. 17 is a box and whisker plot illustrating a degree of dispersion of data shown in FIG. 16.

FIG. 18 is a diagram illustrating an example of an X-ray dynamic image of a chest and is an illustrative diagram illustrating a case where the entire heart and the entire lung field are set as target regions.

FIG. 19 is a graph illustrating signal values of a blood flow waveform in a case where the entire heart is set as a target region.

FIG. 20 is a graph showing a signal value of a blood flow waveform when the entire lung field is set as a target region.

FIG. 21 is a graph showing a relationship between the signal value ratio of the heart and the lung field and the regurgitation rate of pulmonary valve regurgitation when the entire heart and the entire lung field are set as target regions.

FIG. 22 is a box and whisker plot illustrating a degree of dispersion of data shown in FIG. 21.

FIG. 23 is a graph illustrating an integrated signal value of a blood flow waveform in a case where the entire heart is set as a target region.

FIG. 24 is a graph showing an integrated signal value of a blood flow waveform when the entire lung field is set as a target region.

FIG. 25 is a graph showing a relationship between the signal value ratio of the heart and the lung field and the regurgitation rate of pulmonary valve regurgitation when the entire heart and the entire lung field are set as target regions and integrated signal values are used.

FIG. 26 is a box and whisker plot illustrating a degree of dispersion of data shown in FIG. 25.

FIG. 27 is a flowchart illustrating an example of a flow of a dynamic image analysis process in a case where the information on pulmonary valve regurgitation is a determination result of the presence or absence of pulmonary valve regurgitation or the like using skewness and kurtosis.

FIG. 28 is an explanatory diagram showing an example in which a threshold value is set using skewness and kurtosis as parameters, and patients are classified with respect to pulmonary valve regurgitation.

### DETAILED DESCRIPTION OF THE EMBODIMENTS

Below, one embodiment of a dynamic image analysis apparatus (analysis apparatus), a recording medium storing a program, and a dynamic image analysis method according to the present invention will be described with reference to the drawings.

However, the scope of the invention is not limited to the disclosed embodiments.

### [Positioning of Dynamic Image Analysis Apparatus]

A dynamic image analysis apparatus (hereinafter referred to as an “analysis apparatus”) according to the present embodiment acquires an X-ray dynamic image (hereinafter simply referred to as a “dynamic image”) from an image imaging system (an imaging apparatus provided in the image imaging system). Then, the analysis apparatus analyzes the acquired dynamic image, and displays the analysis result to support diagnosis by a doctor or the like.

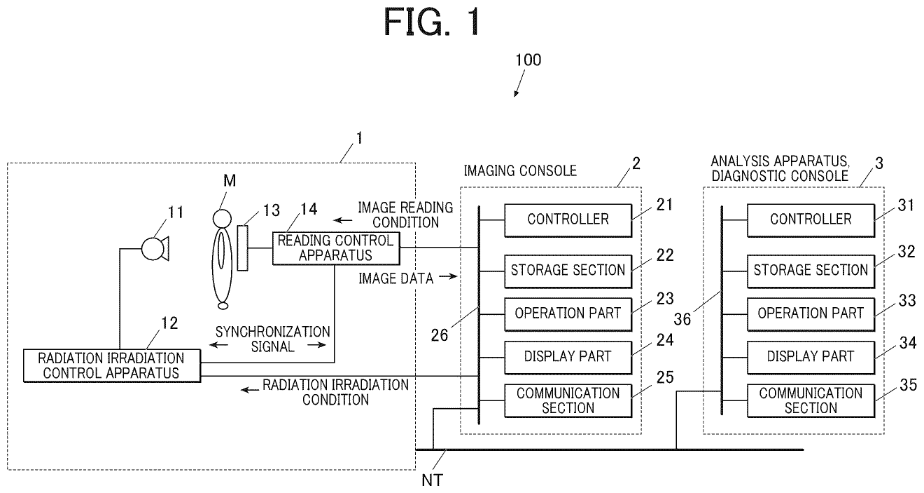

First, as a premise, the relationship between the image imaging system and the analysis apparatus assumed in the present embodiment will be described with reference to FIG. 1.

FIG. 1 shows an overall configuration of an image imaging system 100 according to the present embodiment.

As illustrated in FIG. 1, the image imaging system 100 includes an imaging apparatus 1, and an imaging console 2 connected to the imaging apparatus 1 by a communication cable or the like. The imaging console 2 controls the imaging apparatus 1. The imaging apparatus 1 can image a dynamic image as described later.

The imaging apparatus 1 is connected to a communication network NT such as a local area network (LAN) via the imaging console 2.

Further, the analysis apparatus 3 according to the present embodiment is connected to the image imaging system 100 through the communication network NT. The dynamic image acquired by the imaging apparatus 1 is sent to the analysis apparatus 3 via the imaging console 2.

In the present embodiment, the analysis apparatus (dynamic image analysis apparatus) 3 is, for example, a diagnostic console as shown in FIG. 1. The analysis apparatus (dynamic image analysis apparatus) 3 of the present embodiment generates “information on pulmonary valve regurgitation” as an analysis result based on a dynamic image, and outputs the information. The analysis apparatus 3 may be a diagnostic console or may be provided as an apparatus separate from the diagnostic console.

The apparatuses included in the image imaging system 100 comply with the Digital Image and Communications in Medicine (DICOM) standard, and communication between the apparatuses is performed in accordance with the DICOM standard.

### [Configuration of Imaging Apparatus]

The imaging apparatus 1 can image, for example, a change in the form due to expansion and contraction of the lungs due to respiratory motion, and a dynamic state such as pulsation of the heart. In the “dynamic imaging”, the imaging apparatus 1 performs imaging by repeatedly irradiating a subject M with pulsed radiation such as X-rays at predetermined time intervals (pulse irradiation) or by continuously irradiating the subject M with a low dose of radiation (continuous irradiation). By this imaging, the imaging apparatus 1 acquires a plurality of images showing a dynamic state of the subject M. A series of images obtained by the “dynamic imaging” is referred to as a “dynamic image”. Each of the plurality of images constituting the “dynamic image” is referred to as a frame image. Here, a chest of a subject (a patient or the like) is used as the subject M. The imaging may be performed in a standing position (in a state in which the subject (patient or the like) is standing) or may be performed in a lying position (in a state in which the subject (patient or the like) is lying down).

Note that “dynamic imaging” in the present embodiment includes moving image imaging. The imaging apparatus 1 can also image a still image. However, imaging a still image while displaying a moving image is not included in “dynamic imaging”. Furthermore, the “dynamic image” includes a moving image. However, an image obtained by imaging a still image while displaying a moving image is not included in the “dynamic image”.

The “dynamic imaging” is imaging with low exposure. The “dynamic imaging” imposes a small burden on a subject (patient or the like) and can be suitably used for a simple examination.

A radiation source 11 is placed at a position opposite to the radiation detector 13 with the subject M interposed therebetween. The radiation source 11 irradiates the subject M with radiation (X-rays) under the control of the radiation irradiation control apparatus 12.

The radiation irradiation control apparatus 12 is connected to the imaging console 2. The radiation irradiation control apparatus 12 performs radiation imaging by controlling the radiation source 11 based on the radiation irradiation conditions input from the imaging console 2. The radiation irradiation conditions input from the imaging console 2 include, for example, a pulse rate, a pulse width, a pulse interval, the number of imaging frames per imaging, a value of an X-ray tube current, a value of an X-ray tube voltage, and a type of additional filter. The pulse rate is the number of times of radiation irradiation per second and coincides with a frame rate described later. The pulse width is a radiation irradiation time per radiation irradiation. The pulse interval is a time from the start of one radiation irradiation to the start of the next radiation irradiation and coincides with a frame interval to be described later.

The radiation detector 13 is formed with a semiconductor image sensor such as a flat panel detector (FPD). The FPD has a glass substrate or the like. On the glass substrate, a plurality of detection elements (pixels) are arranged in a matrix at predetermined positions on the substrate. The detection elements (pixels) detect radiation emitted from the radiation source 11 and transmitted through at least the subject M in accordance with the intensity of the radiation. The detection element (pixel) converts the detected radiation into an electric signal and accumulates the electric signal. Each pixel includes a switching unit such as a thin film transistor (TFT). The FPD includes an indirect conversion type and a direct conversion type, and either of them may be used. The indirect conversion type FPD converts X-rays into an electric signal by a photoelectric conversion element through a scintillator. The direct conversion type FPD directly converts X-rays into an electric signal.

A reading control apparatus 14 is connected to the imaging console 2. The reading control apparatus 14 controls a switching unit of each pixel of the radiation detector 13 based on the image reading condition to switch the reading of the electric signal accumulated in each pixel. The image reading conditions are input from the imaging console 2. Then, the reading control apparatus 14 reads the electric signal accumulated in the radiation detector 13 to acquire image data. This image data is each frame image or still image of the dynamic image. If a structure exists between the radiation source 11 and the radiation detector 13, the amount of radiation that reaches the radiation detector 13 decreases due to the structure. For this reason, the signal value (pixel value, density value) of each pixel of the image data changes according to the structure of the subject M. The reading control apparatus 14 outputs the acquired dynamic image or still image to the imaging console 2. The image reading condition is, for example, a frame rate, a frame interval, a pixel size, an image size (matrix size), or the like. The frame rate is the number of frames acquired per second and matches the pulse rate. The frame interval is a time from the start of the acquisition operation of one frame image to the start of the acquisition operation of the next frame image and matches the pulse interval.

Here, the radiation irradiation control apparatus 12 and the reading control apparatus 14 are connected to each other and exchange synchronization signals with each other. Accordingly, the radiation irradiation control apparatus 12 and the reading control apparatus 14 synchronize the radiation emission operation and the image reading operation.

### [Configuration of Imaging Console]

The imaging console 2 outputs the radiation irradiation conditions and the image reading conditions to the imaging apparatus 1. The imaging console 2 controls radiographic imaging and radiographic image reading operations performed by the imaging apparatus 1.

As illustrated in FIG. 1, the imaging console 2 includes a controller 21, a storage section 22, an operation part 23, a display part 24, and a communication section 25, and these components are connected to each other by a bus 26.

The controller 21 includes a central processing unit (CPU), a random access memory (RAM), and the like. The CPU of the controller 21 reads a system program and various processing programs stored in the storage section 22 in response to an operation of the operation part 23 and develops the programs in the RAM. The CPU of the controller 21 executes various processes in accordance with the program expanded in the RAM. Accordingly, the CPU of the controller 21 centrally controls the operation of each unit of the imaging console 2 and the radiation irradiation operation and the reading operation of the imaging apparatus 1.

The storage section 22 is a non-volatile semiconductor memory, a hard disk, or the like. The storage section 22 stores various programs to be executed by the controller 21. In addition, the storage section 22 stores data such as parameters and processing results necessary for executing processing by the program. For example, radiation irradiation conditions and image reading conditions are stored in the storage section 22. The radiation irradiation conditions and the image reading conditions are associated with subject sites. The various programs are stored in the form of readable program codes. The controller 21 sequentially executes operations according to the program code.

The operation part 23 includes a keyboard having cursor keys, character/numeral input keys, various function keys, and the like, and a pointing device such as a mouse. The operation part 23 outputs an instruction signal input by a key operation on the keyboard or a mouse operation to the controller 21. Further, the operation part 23 may include a touch screen on the display screen of the display part 24. In this case, the operation part 23 outputs an instruction signal input via the touch screen to the controller 21.

The display part 24 is a monitor such as a liquid crystal display (LCD). The display part 24 displays an input instruction from the operation part 23, data, and the like in accordance with an instruction of a display signal input from the controller 21.

The communication section 25 includes a LAN adapter, a modem, a terminal adapter (TA), and the like. The communication section 25 controls data transmission and reception to and from each device connected to the communication network NT.

### [Configuration of Analysis Apparatus]

The analysis apparatus 3 is a dynamic image analysis apparatus that acquires a dynamic image (X-ray dynamic image) imaged by the imaging apparatus 1. The analysis apparatus 3 generates “information on pulmonary valve regurgitation” based on the dynamic image information on the “site related to the pulmonary artery” in the dynamic image. The analysis apparatus 3 outputs the generated result to a display part 34 (described later) of the analysis apparatus 3, an external display apparatus (not illustrated), or the like. The analysis apparatus 3 is used as a diagnosis support apparatus for supporting diagnosis by a doctor.

The analysis apparatus 3 is, for example, a computer device such as a personal computer (PC) or a workstation.

As shown in FIG. 1, the analysis apparatus 3 includes a controller 31 (hardware processor), a storage section 32, an operation part 33, a display part 34, and a communication section 35, and each unit is connected by a bus 36.

FIG. 1 illustrates a case in which the analysis apparatus 3, which is a diagnostic console, and the imaging console 2 are separate apparatuses. However, the configuration of the system including the analysis apparatus 3 is not limited thereto. For example, the analysis apparatus 3 which is a diagnostic console may be a multipurpose apparatus having a function as the imaging console 2. In this case, the analysis apparatus 3 and the imaging apparatus 1 may be connected by the communication network NT. In addition, a diagnostic console may be provided separately from the analysis apparatus 3.

The controller 31 (hardware processor) includes a central processing unit (CPU) and a random access memory (RAM) (not illustrated). The CPU of the controller 31 reads a system program and various processing programs stored in the storage section 32 in response to an operation of the operation part 33 and develops the programs in the RAM. Then, the CPU of the controller 31 executes various kinds of processing in accordance with the developed program, and centrally controls the operation of each unit of the analysis apparatus 3.

The storage section 32 is a nonvolatile semiconductor memory, a hard disk, or the like. The storage section 32 is a computer-readable recording medium that stores various programs executed by the controller 31. The storage section 32 stores data such as various programs executed by the controller 31, parameters necessary for execution of processing by the programs, and processing results. The various programs stored in the storage section 32 are stored in the form of readable program codes. The controller 31 sequentially executes operations according to the program code. When the CPU of the controller 31 operates in accordance with the program stored in the storage section 32, the controller 31 performs the following processes. That is, the controller 31 executes a process of acquiring a dynamic image of the chest obtained by dynamic imaging using radiation, for example. Furthermore, the controller 31 executes processing for generating information on pulmonary valve regurgitation, based on dynamic image information on a site related to at least one of the pulmonary artery and the heart in the dynamic image. In addition, the controller 31 executes processing of outputting the generated information on the pulmonary valve regurgitation. Note that details will be given later.

The operation part 33 includes a keyboard having cursor keys, character/numeral input keys, various function keys, and the like, and a pointing device such as a mouse. The operation part 33 outputs an instruction signal input by a key operation on the keyboard or a mouse operation to the controller 31. Further, the operation part 33 may include a touch screen on the display screen of the display part 34. In this case, the operation part 33 outputs an instruction signal input via the touch screen to the controller 31.

The display part 34 is a monitor such as an LCD. The display part 34 performs various displays in accordance with an instruction of a display signal input from the controller 31.

The communication section 35 controls data transmission and reception to and from each device connected to the communication network NT. The communication section 35 includes a LAN adapter, a modem, a TA, and the like.

In the present embodiment, the controller 31 acquires, for example, a dynamic image of the chest via the communication section 35. The dynamic image of the chest is acquired, for example, by the imaging apparatus 1 performing dynamic imaging of the chest of the subject with radiation.

The controller 31 generates “information on pulmonary valve regurgitation”. The “information on pulmonary valve regurgitation” is generated based on dynamic image information on a “site related to the pulmonary artery” (also simply referred to as a “site”) in the dynamic image.

Here, the process in which the controller 31 generates the “information on pulmonary valve regurgitation” will be described in detail.

First, the controller 31 focuses on the “site related to the pulmonary artery” in order to generate the “information on pulmonary valve regurgitation”. The “site related to the pulmonary artery” is a “site” in which a blood flow situation (presence or absence of a regurgitation or the like) in the pulmonary artery can be observed.

FIG. 2 is an illustrative diagram of a dynamic image obtained by X-ray imaging (dynamic imaging) of the chest. In the drawings, a portion surrounded by a one dot chain line indicates a heart. In addition, in the drawing, portions shown in black on the left and right sides indicate the left and right lung fields. The pulmonary artery is an artery that pumps blood out of the heart and into the lungs. The pulmonary artery starts from the pulmonary valve of the right ventricle, passes through the pulmonary trunk, and then branches into two pulmonary arteries (left pulmonary artery and right pulmonary artery) corresponding to the left and right lungs. As illustrated by a broken line in the drawing, the pulmonary trunk is a relatively thick and short portion. The vicinity of the pulmonary trunk before dividing into the right pulmonary artery and the left pulmonary artery is generally referred to as the pulmonary artery origin.

Conventionally, a magnetic resonance imaging (MRI) examination has been performed as a method for diagnosing the state of blood flow in the pulmonary artery (the presence or absence of regurgitation, and the like). In conventional MRI, an examination is performed mainly using the pulmonary artery origin as a target region (region of interest, ROI).

It is desirable that the “site related to the pulmonary artery” in the dynamic image is also a site close to the pulmonary artery origin. However, many structures are gathered around the pulmonary trunk. Therefore, even when radiation (X-ray) imaging is performed around the pulmonary artery trunk, it is difficult to obtain a signal of only the pulmonary artery origin. Therefore, in the present embodiment, as the “site related to the pulmonary artery”, for example, a site that is inside at least one of the right and left lung field regions and includes the right and left pulmonary artery proximal portions is set as a target region (ROI). More preferably, the target regions indicated as “β” and “γ” in FIG. 2 are set as the “sites related to the pulmonary artery”. The target regions indicated as “β” and “γ” in FIG. 2 are regions set inside the left and right lung field regions and in the proximal parts of the left and right pulmonary arteries.

In the present embodiment, “sites related to the pulmonary artery” (target regions “β” and “γ” in the drawing) are set in the right and left lung field regions, respectively, and the state of blood flow in the pulmonary artery (the presence or absence of regurgitation, and the like) is diagnosed. Thus, even in a case where, for example, regurgitation from one of the right and left lungs is slight but regurgitation from the other lung is large, pulmonary valve regurgitation is not overlooked. Note that when “sites related to the pulmonary artery” are set in the right and left lung field regions, respectively, it is preferable to set an average value of values in both of the right and left target regions (in FIG. 2, the target regions “β” and “γ”) as “information on pulmonary valve regurgitation”. By considering the values of both the right and left target regions, it is possible to obtain a result with the same high accuracy as that of, for example, MRI or the like in which the periphery of the pulmonary artery trunk (so-called pulmonary artery origin) is examined as the target region.

In the present embodiment, as described later, a target region (ROI) in a case of extracting information is also set in the heart. The target region set in the heart is “α” in FIG. 2. The waveform differs between an atrium and the ventricle of the heart. According to the present embodiment, a target region (ROI) of the heart is disposed in a portion of the ventricle. The portion of the ventricle where the target region of the heart is located is a portion where the left ventricle and the right ventricle are likely to overlap.

All of the target regions “α”, “β”, and “γ” illustrated in FIG. 2 are, for example, regions having a diameter of about 10 mm. The target region may be automatically set as described later. Further, the target region may be manually set by a user such as a doctor while viewing a screen or the like.

Note that the target region (“site related to the pulmonary artery” or region set in the heart) is not limited to the case of being a target region of about 10 mm in diameter. As described below, the entire lung field or the entire heart may be set as the target region.

Furthermore, the dynamic image information on the “site related to the pulmonary artery” in the dynamic image is, for example, a signal value (a pixel value or the like) of each pixel extracted from the dynamic image. That is, the dynamic image information includes information on the shading (density) of the color and the brightness (luminance) in each pixel in the “site related to the pulmonary artery” in the dynamic image. In this case, a “pixel” from which a signal value is extracted may be a pixel on hardware or a pixel on which various kinds of image processing (e.g., binning processing) have been performed by software.

In the present embodiment, the controller 31 generates “information on pulmonary valve regurgitation” based on information obtained from the signal values of pixels in the “site related to the pulmonary artery”. Note that the controller 31 may generate the “information on pulmonary valve regurgitation” based on the average value of the signal values of the pixels in the “site related to the pulmonary artery”.

The “information on pulmonary valve regurgitation” generated by the controller 31 is, for example, information on whether or not the subject (patient or the like) whose dynamic image has been imaged has pulmonary valve regurgitation. The information on whether or not the subject (patient or the like) has pulmonary valve regurgitation includes information on the presence or absence of pulmonary valve regurgitation and the presence or absence of a possibility of regurgitation. For example, when the value of the dynamic image information is equal to or more than a predetermined reference value, the controller 31 generates “information on pulmonary valve regurgitation” indicating that regurgitation is “present”. In addition, for example, in a case where the value of the dynamic image information is less than a predetermined reference value, the controller 31 generates “information on pulmonary valve regurgitation” indicating that regurgitation is “absent”. Note that the predetermined reference value is set as appropriate.

The “information on pulmonary valve regurgitation” is not limited to information such as the presence or absence of the pulmonary valve regurgitation. The “information on pulmonary valve regurgitation” may be, for example, information related to the severity of pulmonary valve regurgitation when there is pulmonary valve regurgitation. The severity of pulmonary valve regurgitation is information about the degree and level of regurgitation. The information on the severity of the pulmonary valve regurgitation may be, for example, numerical and quantified information such as a regurgitation rate or a prediction value of the regurgitation rate indicating how much blood flows back to the heart among blood pumped from the heart to the lungs.

The “information on pulmonary valve regurgitation” generated by the controller 31 may be information on the shape of the blood flow waveform in the “site related to the pulmonary artery”. The blood flow waveform is a signal waveform that synchronizes with a heart rate. Furthermore, the “information on pulmonary valve regurgitation” may be information obtained by visualizing the blood flow in the “site related to the pulmonary artery”. That is, for example, a color map or the like which visually illustrates the blood flow volume (regurgitation volume) in a target region (ROI) or the like of the lung field may be used. By showing the information obtained by visualizing the blood flow, it is possible for a user such as a doctor to easily understand the medical condition (the presence or absence of the regurgitation, the severity, or the like) of the patient.

In addition, the controller 31 may perform more detailed analysis based on the blood flow waveform in the “site related to the pulmonary artery”. In this case, the controller 31 generates the analysis result as “information on pulmonary valve regurgitation”.

Further, the controller 31 may generate the “information on pulmonary valve regurgitation” based on various indexes (other than the blood flow waveform) obtained in the “site related to the pulmonary artery”. Examples of the various indexes other than the blood flow waveform include a blood flow velocity, a blood flow volume amplitude), and a ratio of signal values (amplitude ratio) of the heart and the lung field. However, various indexes other than the blood flow waveform are not limited thereto.

The details of the “information on pulmonary valve regurgitation” generated by the controller 31 as a generating section will be described later.

Furthermore, the controller 31 outputs the generated “information on pulmonary valve regurgitation” (diagnostic assistance information) to various output destinations.

Note that a destination to which the controller 31 outputs various kinds of information is not particularly limited. For example, the controller 31 may output information to a display part 34 or the like of the analysis apparatus 3. In addition, the controller 31 may output information to various external display devices or various external printers (not shown). The term “output” also includes a case of outputting to an external apparatus as print data or image data. “Output” may be performed via various networks or may be performed by wired or wireless communication. In addition, it may be performed through various connectors, ports of various media, or the like.

### [Operation of Image Imaging System]

Next, operation of the image imaging system 100 will be described.

### (Operation of Imaging Apparatus and Imaging Console)

First, an imaging operation of the imaging apparatus 1 and the imaging console 2 will be described.

The controller 21 of the imaging console 2 sets radiation irradiation conditions in the radiation irradiation control apparatus 12 and sets image reading conditions in the reading control apparatus 14.

Next, the controller 21 outputs an imaging start instruction of the dynamic image to the radiation irradiation control apparatus 12 and the reading control apparatus 14 and controls the imaging of the dynamic image. Specifically, the controller 21 sets a pulse interval at which radiation is emitted in the radiation irradiation control apparatus 12. The radiation irradiation control apparatus 12 directs the radiation source 11 to emit radiation at the set pulse interval. The reading control apparatus 14 outputs the image data (frame image) acquired from the radiation detector 13 to the imaging console 2. When imaging of a predetermined number of frames ends, the controller 21 outputs an instruction to end imaging to the radiation irradiation control apparatus 12 and the reading control apparatus 14 and stops the imaging operation. The controller 21 causes the storage section 22 to store each frame image acquired by imaging. Each frame image is stored in the storage section 22 in association with a number (frame number) indicating an imaging order.

Next, the controller 21 allows the display part 24 to display a dynamic image. When the person performing the imaging confirms the image suitable for the diagnosis, the person performing the imaging inputs a confirmation instruction from the operation part 23. When the confirmation instruction is input by the person performing the imaging, the controller 21 attaches patient information (patient ID of the examinee, a patient name, and the like), examination information, and the like to each of a series of frame images acquired by the dynamic imaging. Then, the controller 21 transmits the dynamic image to the analysis apparatus 3, which is a diagnostic console or the like, through the communication section 25.

### (Operation of Analysis Apparatus)

Next, operation of the analysis apparatus 3 will be described.

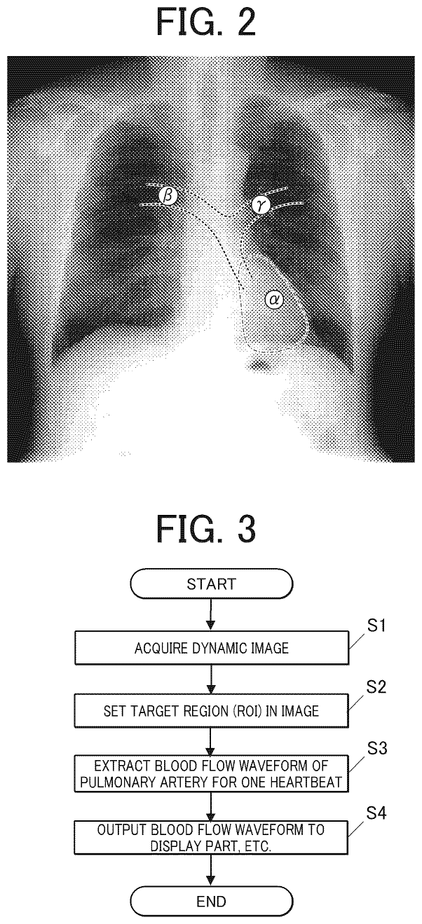

FIG. 3 is a flowchart illustrating an example of dynamic image analysis processing executed by the analysis apparatus 3. The dynamic image analysis processing is processing for supporting a diagnosis using a dynamic image when a doctor makes a diagnosis of pulmonary valve regurgitation. The dynamic image analysis processing is executed by the controller 31 in cooperation with a program stored in the storage section 32.

FIG. 3 illustrates a process in a case where the “information on pulmonary valve regurgitation” is information on the blood flow waveform of the pulmonary artery (information on the shape of the blood flow waveform).

First, when dynamic imaging is performed on the chest of the subject by the imaging apparatus 1, the controller 31 acquires a dynamic image generated by the imaging apparatus 1 (step S1). That is, the controller 31 acquires a dynamic image from the imaging console 2 via the communication section 35. The dynamic image is composed of a plurality of frame images. The controller 31 allows the storage section 32 to store the acquired dynamic image.

For example, FIG. 2 is an illustrative diagram of the dynamic image targeting a chest. FIG. 2 shows an example of the dynamic image imaged in a breath-hold state.

Next, the controller 31 sets target regions “β” and “γ” (step S2). In FIG. 2, the target regions “β” and “γ” are circled portions. The target regions “β” and “γ” are “sites related to the pulmonary artery” and are, for example, the inside of the right and left lung field regions and proximal parts of the right and left pulmonary arteries.

This target region is a portion with a large blood flow and appears whitish in the image. Therefore, the controller 31 automatically sets, for example, target regions “β” and “γ” in regions where many pixels having high luminance values are gathered in the lung field in the dynamic image.

Note that as described above, the target regions “β” and “γ” are not limited to situations in which they are automatically set. The target regions “β” and “γ” may be manually set by a doctor or the like who is a user. In this case, for example, it is preferable to cause the display part 34 or the like to display an image which clearly illustrates the contrast between a portion which appears white due to the blood flow and the other portions. Thus, it is possible to guide the setting position of the target region and to support the setting by the user. Further, in a case where the position of the target region set by the user is largely deviated from the position to be originally set, the user may be requested to correct the set position. When the user is requested to correct the set position, for example, an error message or an alert is output. In this case, a position to be set as the target region may be presented to the user by displaying a marker or the like at a desirable setting position.

When a signal value or the like of the pixel is extracted from the “site related to the pulmonary artery”, various kinds of noise are superimposed on the signal value. Therefore, it is difficult for the controller 31 to acquire only necessary information from the signal values of the pixels and the like.

Therefore, according to the present embodiment, a target region (“α” surrounded by a circle in FIG. 2) is also set in the heart. Next, the controller 31 measures the frequency of a luminance change due to the pulsation of the heart from the signal values of the pixels in the target region (“α”) and acquires the frequency of the heart rate. Furthermore, the controller 31 generates a band-pass filter corresponding to the frequency of the heart rate. By applying this band pass filter to the values detected from the target regions (“β” and “γ” in FIG. 2) set as the “site related to the pulmonary artery”, the controller 31 can obtain a value in a state where noise other than the necessary signal is reduced.

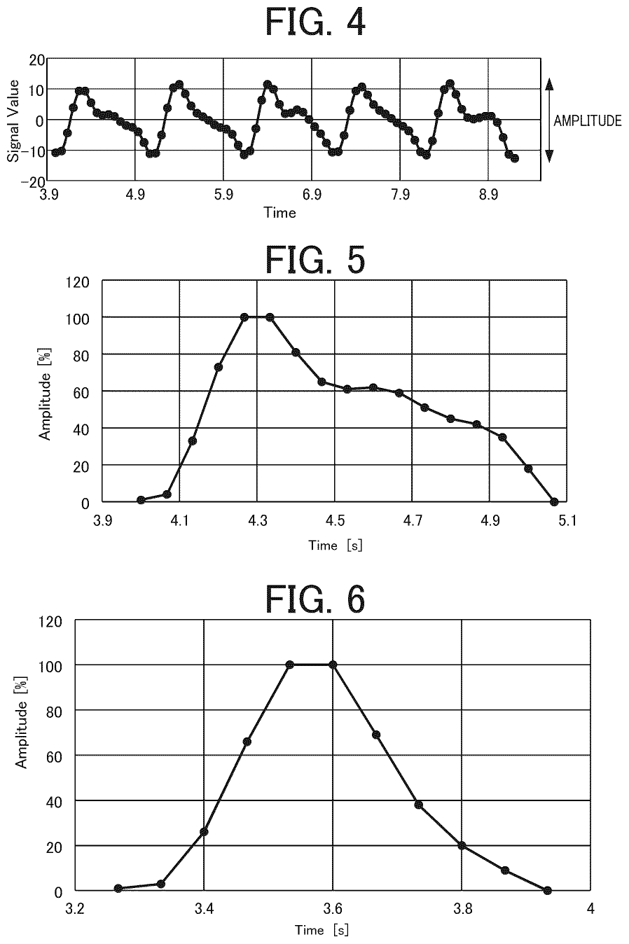

FIG. 4 is an example of a blood flow waveform detected from “β” and “γ” in FIG. 2. “β” and “γ” are the target regions set in the left and right pulmonary artery proximal portions inside the left and right lung field regions as the “site related to the pulmonary artery”. In FIG. 4, the waveform is obtained after applying a band-pass filter based on information obtained from the target region (“α” in FIG. 2) set in the heart. In addition, the minimum value (approximately “−10”) of the amplitude of the blood flow waveform is set as the start of the wavelength.

Generally, the heart beats about once a second. In the dynamic imaging, imaging is performed for about 5 seconds to 15 seconds. Therefore, it is possible to obtain the blood flow waveform of the pulmonary artery for about five to ten cycles of the pulsation (heartbeat) of the heart by a series of dynamic imaging. FIG. 4 illustrates a blood flow waveform for five cycles.

The shape of the blood flow waveform is seen from the blood flow waveform of the pulmonary artery for one heartbeat. Therefore, the controller 31 extracts the blood flow waveform of the pulmonary artery for one heartbeat from the blood flow waveform detected from the target regions (“β” and “γ”) (step S3, refer to FIG. 5). Note that the blood flow waveform of the pulmonary artery for one heartbeat may be an average value of a plurality of waveforms (blood flow waveforms for five heartbeats in FIG. 4) as shown in FIG. 4. Note that the controller 31 may extract an average value of data obtained from the target regions “β” and “γ” instead of the blood flow waveform detected from the target regions (“β” and “γ”).

In a case in which the blood flow waveform of the pulmonary artery for one heartbeat is extracted, the controller 31 may perform normalization such that each of the signal values forming the blood flow waveform takes a value in a certain range. The value in the certain range is, for example, a value in which maximum value-minimum value is 0-100. FIG. 5 and FIG. 6 show graphs when the signal values are normalized.

In a normal state where there is no pulmonary valve regurgitation, for example, when it is necessary to send “100” of blood from the heart to the lungs, “100” of blood is sent from the heart. The blood pumped out from the heart flows into the lungs at once and then flows out gradually. Therefore, the peak comes at a temporally early stage. Looking at this in the graph shown in FIG. 5, the peak is closer to the left side than the center. In the case of a normal state in which there is no regurgitation or the like, the gradient of the decrease in blood flow is also gentle. Then, as illustrated in the graph of FIG. 5, the blood flow waveform has a gradually decreasing waveform. For example, FIG. 5 shows an example of a blood flow waveform in a case of a person (patient) with a regurgitation rate of 0. In contrast, for example, in the case of a person who has 20% pulmonary valve regurgitation, when it is necessary to send “100” of blood from the heart to the lungs, “120” of blood is sent out from the heart and flows into the lungs. This is because the amount of blood that is returned to the heart by regurgitation is added. In this case, since the amount of blood flowing in is large, the inflow time until reaching the peak becomes long. Looking at this in the graph shown in FIG. 6, the peak is closer to the center than in the case of FIG. 5. Thereafter, the gradient of the blood flow waveform after passing through the peak becomes steep due to the influence of the blood outflow corresponding to the regurgitation. Then, as illustrated in the graph of FIG. 6, the blood flow waveform becomes a waveform that rapidly decreases. For example, FIG. 6 shows an example of a blood flow waveform in a case of a person (patient) with a regurgitation rate of 54.5.

After extracting the shape of the blood flow waveform in the pulmonary artery, the controller 31 outputs the blood flow waveform to the display part 34 or the like (step S4). Accordingly, the blood flow waveforms as illustrated in FIG. 5 and FIG. 6 are displayed on the display part 34. A doctor or the like who is a user can check the shape of the waveform on the display screen.

If the blood flow waveform displayed on the display part 34 has a shape close to the shape shown in FIG. 5, the user determines (diagnoses) that the person (patient) has no symptom of pulmonary valve regurgitation (or has a high possibility of having no pulmonary valve regurgitation). If the blood flow waveform displayed on the display part 34 has a shape close to the shape shown in FIG. 6, it is determined (diagnosed) that the person (patient) has a symptom of pulmonary valve regurgitation (or is highly likely to have pulmonary valve regurgitation).

In a case where it is determined from the shape of the blood flow waveform that there is a symptom of pulmonary valve regurgitation or there is a high possibility of pulmonary valve regurgitation, it is considered that a doctor who is a user instructs the patient to further perform a detailed examination such as an MRI examination.

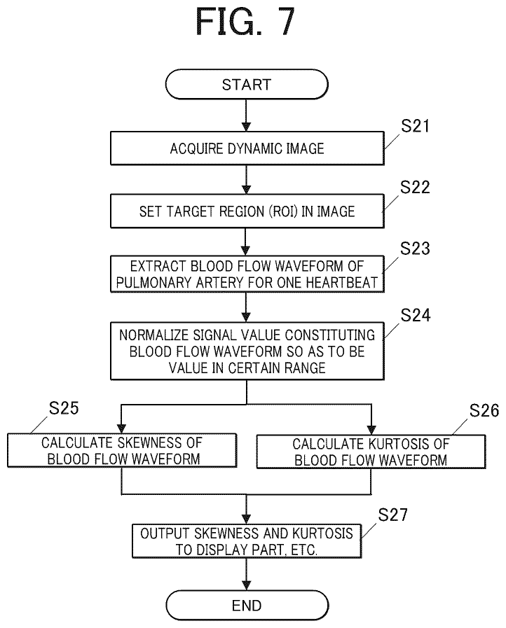

Next, the example shown in FIG. 7 shows processing in the case where the “information on pulmonary valve regurgitation” is a parameter relating to the shape of the blood flow waveform of the pulmonary artery.

Here, the “parameter regarding the shape of the blood flow waveform of the pulmonary artery” is, for example, “skewness”, “kurtosis”, or the like.

In this case, first, the controller 31 extracts the blood flow waveform of the pulmonary artery for one heartbeat on the basis of the information obtained from the target regions (“β” and “γ” in FIG. 2). Thereafter, the controller 31 normalizes each of the signal values constituting the blood flow waveform so as to be a value in a certain range (step S24). The value in the certain range is, for example, a value in which maximum value-minimum value is 0-100. Note that step 21 to step S23 in FIG. 7 are the same as step S1 to step S3 in FIG. 3, and hence description thereof is omitted.

Further, the controller 31 calculates a “skewness” and a “kurtosis” as parameters related to the shape of the blood flow waveform (step S25 and step S26). Note that the calculation of “skewness” and “kurtosis” may be performed in parallel as shown in FIG. 7. Alternatively, the calculations of the “skewness” and the “kurtosis” may be sequentially performed such that one is calculated first and then the other is calculated.

The “skewness” can be calculated by the following formula 1.

Note that in formula 1, “n” represents a sample amount, “xi” represents sample data, x bar represents an average value, and “s” represents a standard deviation.

$$\frac{n}{( n - 1 ) ( n - 2 )} {\sum }_{i = 1}^{n} {( \frac{{x}_{i} - \overline{x}}{s} )}^{3} \qquad [ Formula 1 ]$$

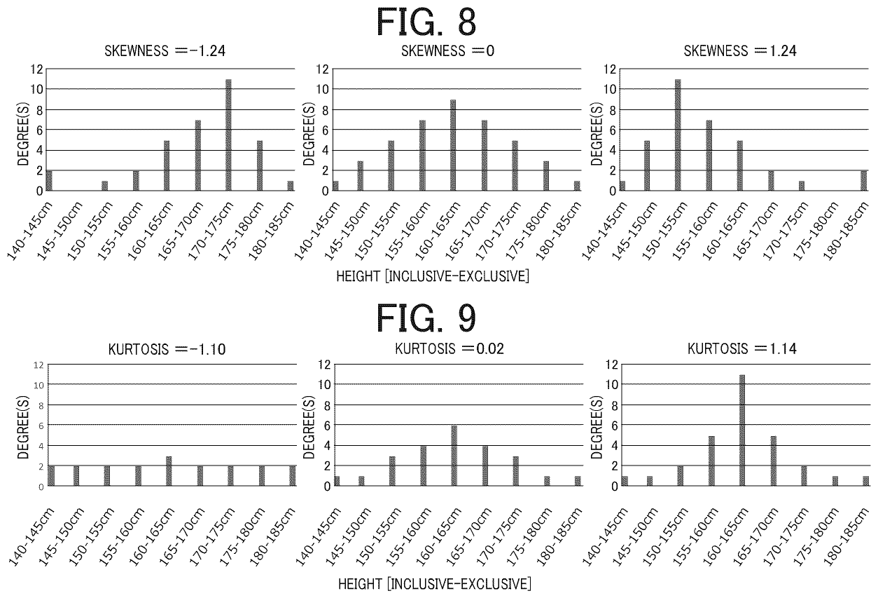

FIG. 8 is a graph illustrating the calculated “skewness”.

The “skewness” is a degree of deviation of a peak when a blood flow waveform is viewed in time series. The blood flow waveform indicates a change in a signal value of a blood flow volume. In the case of “skewness=0” shown in the center of FIG. 8, that is, in the case where there is no “skewness”, the peak of the blood flow rate comes to substantially the center of the blood flow waveform. The blood flow waveform in this case is close to the graph shape of FIG. 6. In addition, in the case of “skewness=1.24” shown on the right side in FIG. 8, the peak of the blood flow amount is closer to the left side than the center of the blood flow waveform, and the blood flow amount gradually decreases after reaching the peak. The blood flow waveform in this case is close to the graph tendency of FIG. 5. Furthermore, in the case of “skewness=−1.24” illustrated on the left side in FIG. 8, a tendency opposite to that in the diagram on the right side of FIG. 8 is illustrated. That is, in the case of “skewness=−1.24”, the peak of the blood flow rate is closer to the right side than the center of the blood flow waveform, and the blood flow rate sharply decreases after reaching the peak.

As shown in FIG. 6 and FIG. 8, as the “skewness” is closer to 0, the degree of the regurgitation rate of the pulmonary artery tends to be higher. Furthermore, as the regurgitation rate of the pulmonary artery decreases, the “skewness” tends to increase in the positive (+) direction. Therefore, by calculating the “skewness”, the degree (level or severity) of the regurgitation rate can be digitized and indicated to the user such as the doctor as a quantitative value. By quantifying the degree of the regurgitation rate, it is possible to provide a user with quantitative information that contributes to the diagnosis of pulmonary valve regurgitation.

The “kurtosis” can be calculated by the following Formula 2.

Note that in Formula 2, “n” represents a sample amount, “xi” represents sample data, x bar represents an average value, and “s” represents a standard deviation.

$$\frac{n ( n + 1 )}{( n - 1 ) ( n - 2 ) ( n - 3 )} {\sum }_{i = 1}^{n} \frac{{( {x}_{i} - \overline{x} \}}^{4}}{{s}^{4}} - \frac{3 {( n - 1 )}^{2}}{( n - 2 ) ( n - 3 )} \qquad [ Formula 2 ]$$

FIG. 9 is a diagram illustrating the calculated “kurtosis”.

As illustrated in FIG. 9, a higher “kurtosis” indicates a steeper change in the signal value of the blood flow waveform. In FIG. 9, the “kurtosis” increases toward the right side.

As shown in FIG. 5 and FIG. 9, as the “kurtosis” is higher, the degree of the regurgitation rate of the pulmonary artery tends to be higher. Therefore, by calculating the “kurtosis”, the degree (level or severity) of the regurgitant rate can be digitized and indicated to a user such as a doctor as a quantitative value. By quantifying the degree of the regurgitation rate, it is possible to provide the user with quantitative information that contributes to the diagnosis of pulmonary valve regurgitation.

Further, the controller 31 may obtain the “amplitude” of the blood flow waveform (see FIG. 4) as the “parameter related to the shape of the blood flow waveform of the pulmonary artery”. The “amplitude” of the blood flow waveform tends to be larger, and the height of the amplitude tends to be higher in a person having a high regurgitation rate. Therefore, also by looking at the “amplitude” of the blood flow waveform, it is possible to determine the presence or absence of the pulmonary valve regurgitation, and the possibility, degree, and the like thereof.

As described above, when each of the signal values of the blood flow waveform is normalized to take a value in a certain range (for example, a value in which maximum value-minimum value is 0 to 100), it is not possible to make a difference in the value of the “amplitude”. Therefore, in a case in which the “amplitude” of the signal value of the blood flow waveform is obtained, the controller 31 extracts the “amplitude” without performing normalization.

As described above, in the present embodiment, a case where the controller 31 can acquire the “skewness”, the “kurtosis”, and the “amplitude” of the blood flow waveform as the “parameter related to the shape of the blood flow waveform of the pulmonary artery” will be exemplified. The controller 31 may acquire all of these or may acquire only a part of these. The controller 31 may acquire various indexes other than these three parameters.

In addition, in a case where the controller 31 obtains “parameters related to the shape of the blood flow waveform of the pulmonary artery” as the information obtained from the signal values of the pixels in “the site related to the pulmonary artery”, the controller 31 may further perform detailed analysis using these parameters and generate “information on pulmonary valve regurgitation”. The “parameter related to the shape of the blood flow waveform of the pulmonary artery” is, for example, “skewness”, “kurtosis”, or “amplitude” of the blood flow waveform.

For example, the controller 31 may perform the multiple regression analysis using three parameters of “skewness”, “kurtosis”, and “amplitude”. In this case, for example, the controller 31 sets a threshold value in advance, and determines that there is pulmonary valve regurgitation or there is no pulmonary valve regurgitation depending on whether or not the threshold value is exceeded. Then, it is considered that the controller 31 sets the determination result as the “information on pulmonary valve regurgitation”. Pulmonary valve regurgitation is not immediately a health problem as long as it is as slight as about several percent and can occur even in healthy individuals. For this reason, the controller 31 should determine that there is pulmonary valve regurgitation only in a case where the regurgitation rate is a pathological level. The threshold value for whether or not the controller 31 determines that there is pulmonary valve regurgitation is, for example, a regurgitation rate of 25%. Whether or not the controller 31 determines that there is pulmonary valve regurgitation when the regurgitation rate is exactly 25% is set as appropriate.

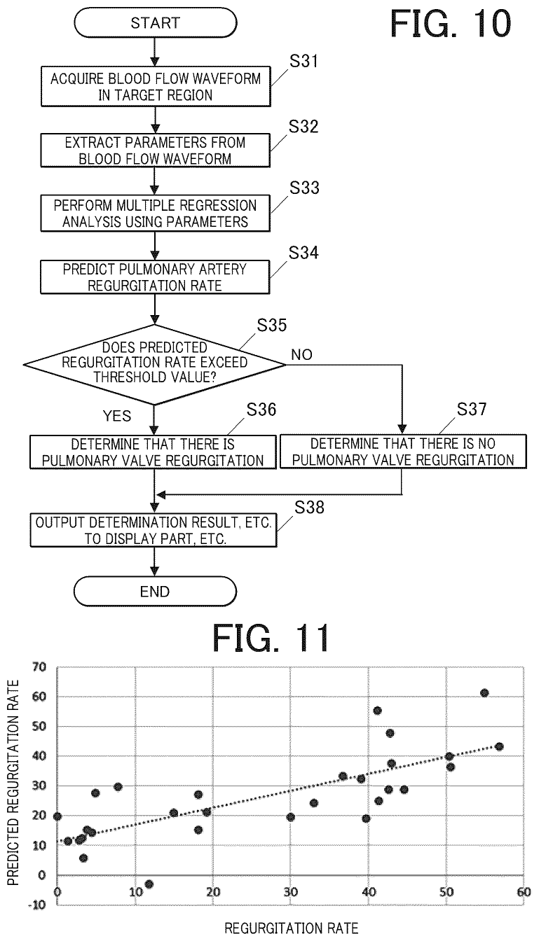

For example, when the controller 31 performs multiple regression analysis using three parameters (variables) of “skewness”, “kurtosis”, and “amplitude”, it is possible to predict the regurgitation rate from these parameters. In this case, for example, as illustrated in FIG. 10, first, the controller 31 acquires the blood flow waveform in the target region (e.g., “β” and “γ” in FIG. 2) that is the “site related to the pulmonary arteries” (step S31). Then, the controller 31 extracts three parameters of “skewness”, “kurtosis”, and “magnitude” from the blood flow waveform (step S32). Further, the controller 31 performs multiple regression analysis using these parameters (variables) (step S33) and predicts the pulmonary artery regurgitation rate (step S34). Then, the controller 31 determines whether or not the derived predicted regurgitation rate exceeds a predetermined threshold value of the regurgitation rate (step S35). When the regurgitation rate exceeds the threshold value (step S35; YES), the controller 31 determines that there is pulmonary valve regurgitation (step S36). On the other hand, when the regurgitant rate is equal to or lower than the threshold value (step S35; NO), the controller 31 determines that there is no pulmonary valve regurgitation (step S37). That is, for example, in a case where the threshold value of the regurgitation rate is 25% as described above, when the predicted regurgitation rate is less than 25%, such as 0% or 10%, the controller 31 determines that the person (patient) does not have pulmonary valve regurgitation (at a pathological level). On the other hand, in a case where the percentage exceeds 25%, such as 30%, the controller 31 determines that there is pulmonary valve regurgitation in the person (patient).

In a case where the determination result related to the predicted regurgitation rate and the pulmonary valve regurgitation corresponding thereto is output, the controller 31 outputs the determination result and the derived regurgitation rate (predicted regurgitation rate) to the display part 34 or the like (step S38) and displays them.

A user such as a doctor can appropriately respond by viewing the result output to the display part 34 or the like. For example, when the controller 31 determines that the patient has pulmonary valve regurgitation or when the predicted regurgitation rate is high, the user instructs, for example, an MRI examination or the like in order to check the situation in more detail.

Even in a case where the predicted regurgitation rate derived by the controller 31 does not exceed the threshold value of the regurgitation rate, when a numerical value close to the threshold value is obtained, an instruction to perform an MRI examination or the like may be issued as a precaution. In addition, pulmonary valve regurgitation is likely to appear after surgery or the like in patients with tetralogy of Fallot. For this reason, the predicted regurgitation rate or the like may be periodically derived in post-operative follow-up observation or the like. In such a case, when there is a change in the derived numerical value, even if it does not exceed a threshold value, it may be instructed to perform a further detailed examination such as an MRI examination.

FIG. 11 is a graph example showing a result of multiple regression analysis using three variables.

FIG. 11 is a graph obtained by plotting the analysis results and drawing a straight line (regression line) so as to best fit each data. A regression equation is obtained by expressing this by a linear function. “r” is an index indicating to what extent the regression equation obtained as a result of the regression analysis can explain the value variation of the predicted regurgitation rate as the objective variable. It can be said that the closer “r” is to “1”, the more accurate the prediction is. FIG. 11 illustrates the case of “r=0.754”. The result shown in FIG. 11 means that the regurgitation rate can be predicted with considerable accuracy.

The parameters used for the prediction of the regurgitation rate by the multiple regression analysis are not limited to the three parameters of “skewness”, “kurtosis”, and “amplitude”. A part of the parameters of “skewness”, “kurtosis”, and “amplitude” may be used for the prediction of the regurgitation rate by the multiple regression analysis. In addition, parameters other than the “skewness”, the “kurtosis”, and the “amplitude” may be included in the parameters used for the prediction of the regurgitation rate by the multiple regression analysis.

In addition, as described above, in the case of a person with pulmonary valve regurgitation, although “100” of blood should be sent from the heart to the lungs, there is a tendency to send “120” or “150” of blood by adding the amount of regurgitation. For this reason, when viewed in a dynamic image, it is assumed that, for example, the ratio between the “amplitude” of the signal value of a pixel that changes in the heart at the time of beating and the “amplitude” of the signal value of a pixel that changes in the lung field is different between a healthy person without regurgitation and a patient with pulmonary valve regurgitation.

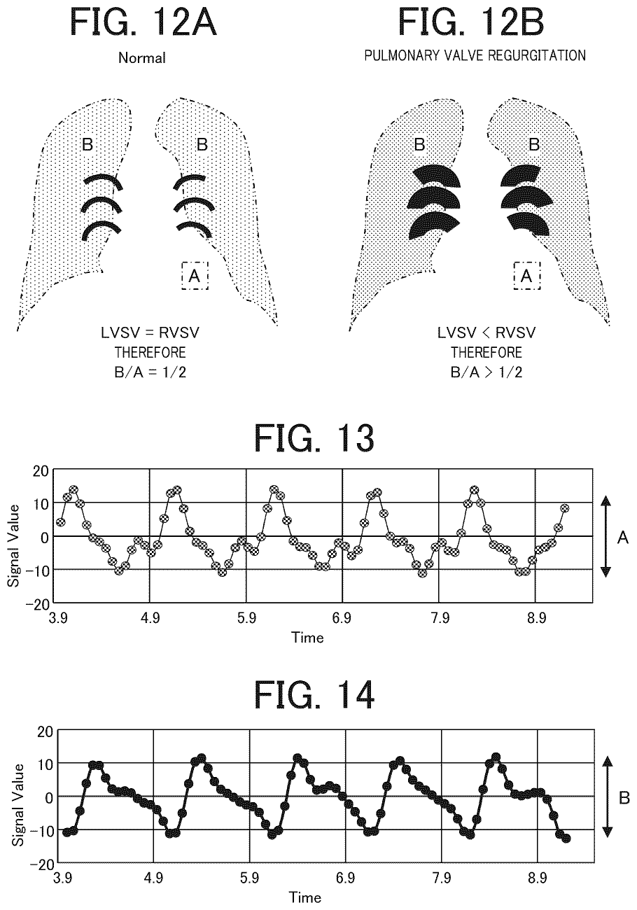

FIG. 12A is an illustrative diagram illustrating blood flow in lung fields of a healthy subject. FIG. 12B is an illustrative diagram illustrating blood flow in the lung fields of a patient with pulmonary valve regurgitation. FIG. 13 shows a change in the signal value of the target region (ROI) set in the heart which is the region indicated by “α” in FIG. 2. The “amplitude” of the signal value illustrated in FIG. 13 is referred to as an “amplitude A”. FIG. 14 shows the change in the signal value of the target region (ROI) set in the lung field which is the region indicated by “β” and “γ” in FIG. 2. The “amplitude” of the signal value illustrated in FIG. 14 is referred to as “amplitude B”. Both the “amplitude A” shown in FIG. 13 and the “amplitude B” shown in FIG. 14 are obtained by removing noise by applying a band-pass filter corresponding to the frequency of the heart rate of the heart.

The amount of change in the “amplitude A” of the signal value in the heart is a single stroke volume LVSV of the left ventricle+a single stroke volume RVSV of the right ventricle. The amount of change in the “amplitude B” of the signal value in the lung field is the one stroke volume RVSV of the right ventricle.

As shown in FIG. 12A, in the case of a healthy person, it is the LVSV=RVSV, which is B/A=1/2. On the other hand, in the case of a patient with regurgitation, a large amount of blood is sent to the lungs as described above. Therefore, as shown in FIG. 12B, LVSV<RVSV, and B/A>1/2.

Such a relationship is recognized between the “amplitude A” of the signal value in the heart and the “amplitude B” of the signal value in the lung field. Therefore, the controller 31 can obtain information (“information on pulmonary valve regurgitation”) indicating the degree of the regurgitation rate (the severity of pulmonary valve regurgitation) by calculating and comparing the ratio between the “amplitude A” of the signal value in one heartbeat of the heart and the “amplitude B” of the signal value corresponding to one heartbeat of the lung field.

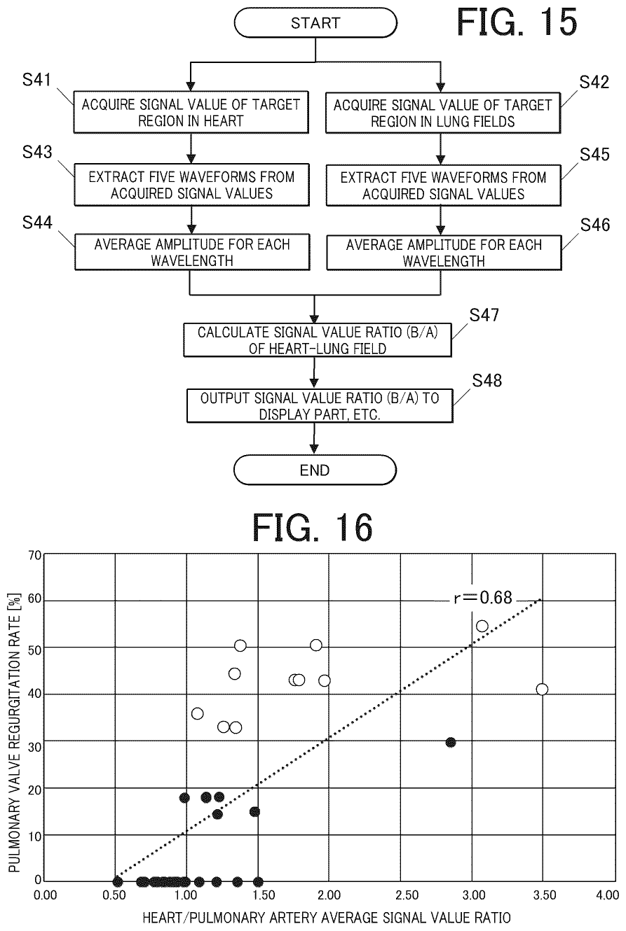

In a case where the controller 31 generates the “information on pulmonary valve regurgitation” from the ratio of the “amplitude A” of the signal value in the heart and the “amplitude B” of the signal value in the lung field, for example, processing illustrated in FIG. 15 is performed.

That is, as shown in FIG. 15, the controller 31 acquires signal values of pixels from the target region (ROI) set in the heart (step S41). In FIG. 2, the target region set in the heart is indicated by “α”. Furthermore, signal values of pixels are acquired from the target regions (ROIs) set in the right and left lung fields (step S42). In FIG. 2, the target regions set in the lung field are denoted by “β” and “γ”.

Furthermore, the controller 31 extracts, from among the signal values of the blood flow waveform acquired from the target region “α” of the heart, five consecutive wavelengths in which the waveform shape is not broken (step S43). Next, the controller 31 averages the amplitudes for each wavelength to obtain “amplitude A” (step S44). Similarly, for the lung fields, the controller 31 extracts five continuous wavelengths in which the waveform shape is not broken from the signal values of the blood flow waveforms acquired from the target regions “β” and “γ” of the lung fields (step S45). Next, the controller 31 averages the amplitudes for each wavelength to obtain “amplitude B” (step S44). The order of the processing of step S41 (and step S43, step S44) for the signal value of the heart and the processing of step S42 (and step S45, step S46) for the signal value of the lung field does not matter.

Next, the controller 31 calculates the signal value ratio (amplitude B/amplitude A) of the heart-lung field (step S47). In a case where the signal value ratio (amplitude B/amplitude A) of the heart-lung field is calculated, the controller 31 outputs the signal value ratio to the display part 34 or the like (step S48) and displays the signal value ratio.

Thus, the controller 31 of the analysis apparatus 3 can provide a doctor or the like who is a user with the “information on pulmonary valve regurgitation” to support a diagnosis. The “information on pulmonary valve regurgitation” is information such as whether or not there is the pulmonary valve regurgitation and how severe the symptom is (the severity of the regurgitation) in a case where there is the pulmonary valve regurgitation.

Note that the relation between the signal value ratio (amplitude B/amplitude A) of the heart-lung field and the pulmonary valve regurgitation rate is verified in association with the regurgitation rate actually confirmed by an MRI examination or the like, and it is recognized that there is a significant correspondence relation.

For example, FIG. 16 is a graph in which the horizontal axis represents the average signal value ratio (amplitude B/amplitude A) of the heart-lung field and the vertical axis represents the pulmonary valve regurgitation rate (%) actually confirmed by an MRI examination or the like.

As illustrated in FIG. 16, it is understood that the value of the average signal value ratio (amplitude B/amplitude A) between the heart-lung field tends to be larger in a person having a higher pulmonary valve regurgitation rate (%) (having a more severe regurgitation symptom).

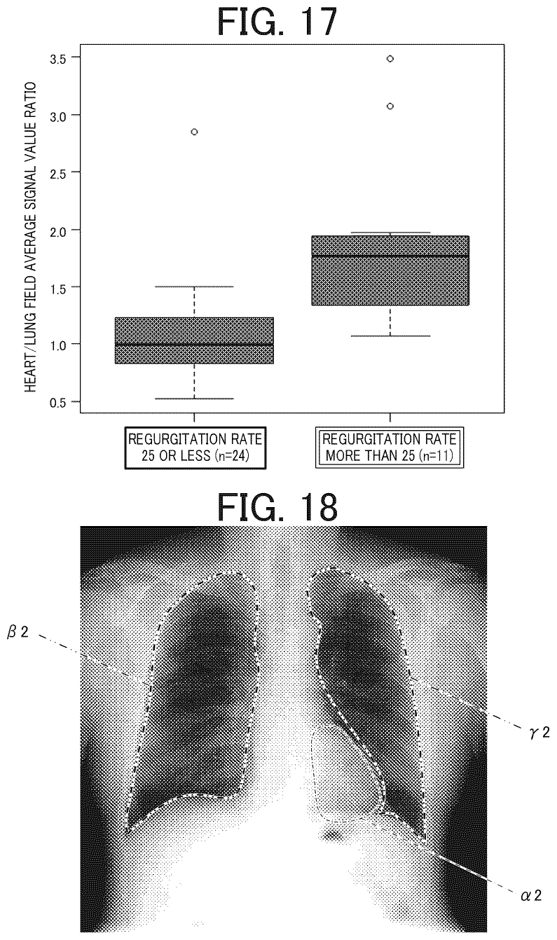

FIG. 17 is a box-and-whisker plot illustrating variations in the data illustrated in FIG. 16.

The left side of FIG. 17 illustrates a group in which the regurgitation rate is 0% to 25% (inclusive), that is, a data group (note that the number of samples n=24) indicated by black dots in FIG. 16. The right side of FIG. 17 is a group of regurgitation rates of more than 25% and shows a data group (number of samples n=11) indicated by white dots in FIG. 16.

Points (singular points) shown in FIG. 17 that are one on the top left side and two on the top right side in FIG. 17 mean data deviated from the range of the average spread of each data group. There are various factors that cause data corresponding to the singular point in the sample. For example, a case where a body motion is large at the time of imaging, a case where the shape of a lung field is peculiar and correct data cannot be obtained, or the like may be determined as noise. On the other hand, when data corresponding to a singular point is generated, there is a possibility that there is a circumstance to be considered. In the present embodiment, the average value and the like are calculated including such a singular point. Also, in FIG. 16 and FIG. 17, a singular point is shown as one of sample data. How to handle the singular point is not limited to the example shown here. For example, when it is known that the reason why the data corresponding to the singular point is generated is simply due to an error at the time of imaging, the data corresponding to the singular point may be excluded from the sample data shown in FIG. 16 and FIG. 17. Further, data such as an average value may be calculated by excluding data corresponding to a singular point.

The case where the controller 31 calculates the heart-lung field average signal value ratio (amplitude B/amplitude A) on the basis of the signal values of the blood flow waveforms acquired from the target region “α” which is a region about 10 mm in the heart and the target regions “β” and “γ” which are regions about 10 mm in the lung field has been described above. However, the average signal value ratio (amplitude B/amplitude A) of the heart-lung field is not limited to the case based on the signal values of the blood flow waveforms acquired from the target regions “α”, “β”, and “γ”. For example, the controller 31 may extract signal values using the entire heart and the entire lung field as the target regions.

FIG. 18 is a diagram illustrating a case where the entire heart and the entire lung field are set as the target regions in a dynamic image. In FIG. 18, the heart region is surrounded by a fine one dot chain line, and each of the left and right lung field regions is surrounded by a two dot chain line.

In this case, the controller 31 extracts five consecutive wavelengths whose shapes are not broken from among signal values of a plurality of blood flow waveforms in the entire heart that is the target region. The target region (ROI) in this case is denoted by “α2” in FIG. 18. Then, the controller 31 averages the amplitude for each wavelength. For the lung field as well, the controller 31 extracts five continuous wavelengths whose shapes are not collapsed from the signal values of the plurality of blood flow waveforms in the entire lung field as the target region. The target region (ROI) in this case is set as “β2” and “γ2” in FIG. 18. Then, the controller 31 averages the amplitude for each wavelength.

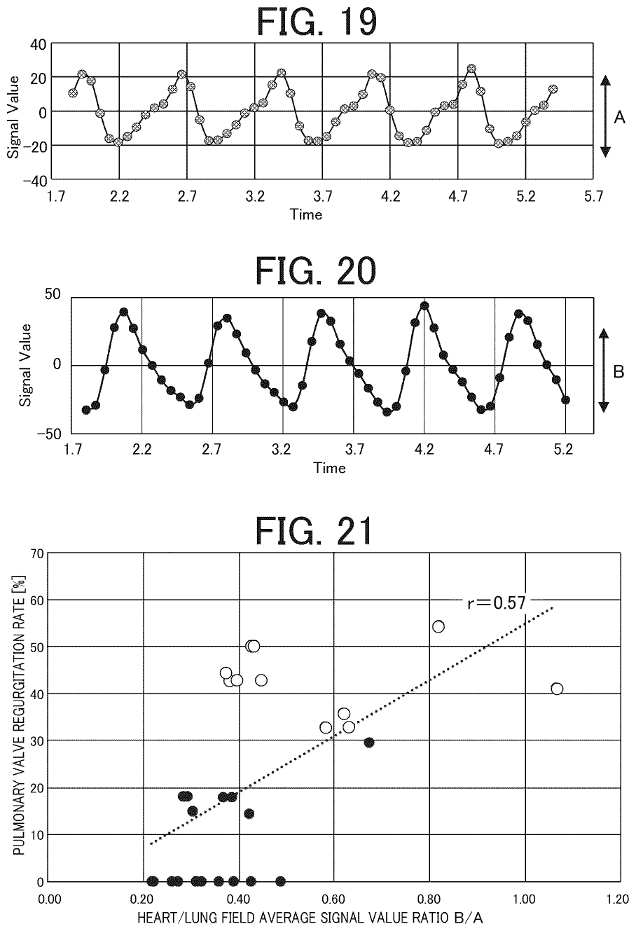

FIG. 19 shows a change in signal value when the entire heart is set as the target region (ROI). The “amplitude” of the averaged signal value is referred to as an “amplitude A”. FIG. 20 shows a change in signal value when the entire lung field is set as the target region (ROI). The “amplitude” of the averaged signal value is referred to as an “amplitude B”. Both the “amplitude A” shown in FIG. 19 and the “amplitude B” shown in FIG. 20 are obtained by removing noise by applying a band-pass filter corresponding to the frequency of the heart rate of the heart.

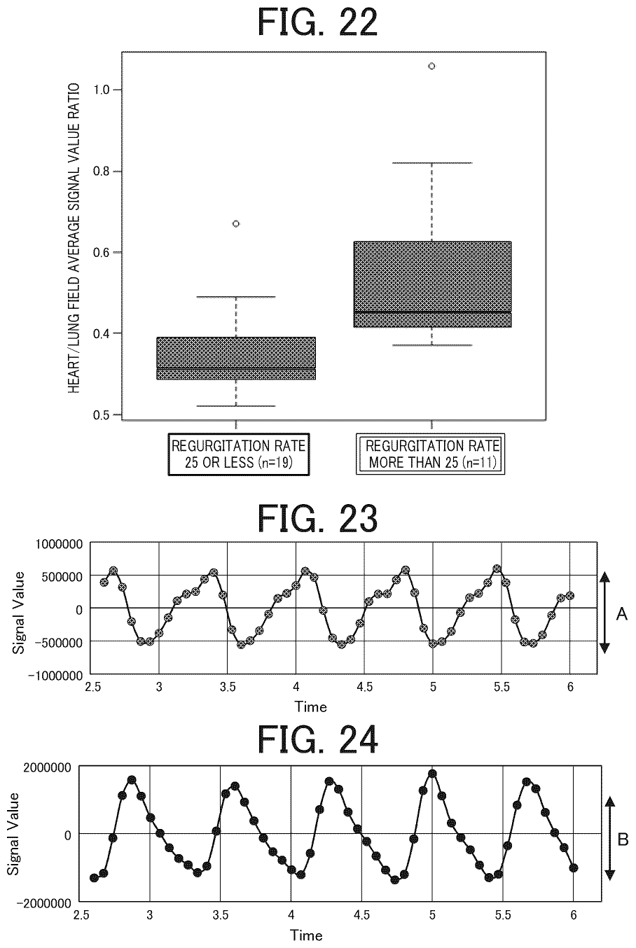

FIG. 21 is a graph illustrating the result of calculating the average signal value ratio (amplitude B/amplitude A) between the heart-lung field by using the “amplitude A” and the “amplitude B” determined as described above. FIG. 22 is a box and whisker plot diagram showing a variation degree of the data shown in FIG. 21.

The content represented by the box and whisker plot and the handling of the singular points are the same as those in FIG. 17 described above, and hence description thereof is omitted.

The controller 31 acquires “amplitude A” and “amplitude B” of the signal value for, for example, the entire heart (“α2” in FIG. 18) and the entire lung field (“β2” and “γ2” in FIG. 18), respectively. Then, the controller 31 obtains the average signal value ratio (amplitude B/amplitude A) of the heart-lung field. Also in this case, it is understood that the value of the average signal value ratio (amplitude B/amplitude A) between the heart-lung field tends to be larger for a person having a higher pulmonary valve regurgitation rate (%) (having a more serious regurgitation symptom).

Further, for example, the controller 31 extracts five consecutive wavelengths whose shapes are not collapsed for the integrated signal value in the entire heart (“α2” in FIG. 18) which is the target region and the integrated signal value in the entire lung field (“β2” and “γ2” in FIG. 18) which is the target region. Next, the controller 31 may calculate “amplitude A” and “amplitude B” by averaging the amplitudes for each wavelength.

FIG. 23 illustrates the change in the integrated signal values in a case where the entire heart is set as the target region (ROI), and the “amplitude” of the signal value obtained by averaging the change in the integrated signal values is set as the “amplitude A”. FIG. 24 illustrates the change in the integrated signal values in a case where the entire lung field is set as the target region (ROI), and the “amplitude” of the signal value obtained by averaging the change in the integrated signal values is set as the “amplitude B”. It should be noted that the “amplitude A” shown in FIG. 23 and the “amplitude B” shown in FIG. 24 are both obtained by removing noise by applying a band-pass filter corresponding to the frequency of the heart rate of the heart.

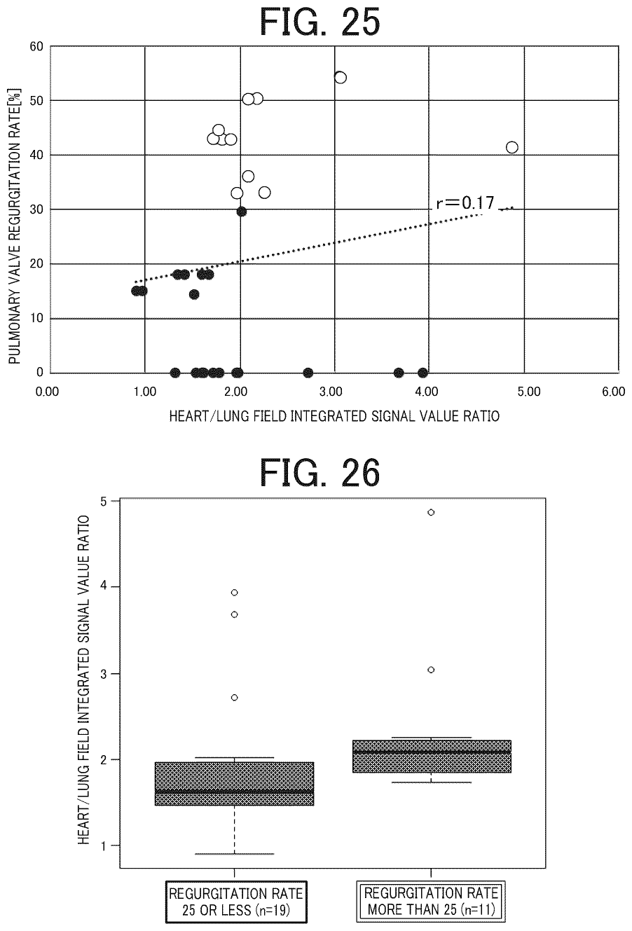

FIG. 25 is a graph illustrating the average signal value ratio (amplitude B/amplitude A) of the heart-lung field calculated using the “amplitude A” and the “amplitude B” obtained as described above. FIG. 26 is a box-and-whisker plot showing variations in the data shown in FIG. 25.

The content represented by the box and whisker plot and the handling of the singular points are the same as those in FIG. 17 described above, and hence description thereof is omitted.

In this way, the controller 31 acquires the “amplitude A” and the “amplitude B” of the integrated signal value for the entire heart (“α2” in FIG. 18) and the entire lung field (“β2” and “γ2” in FIG. 18), respectively. Even in a case where the controller 31 obtains the average signal value ratio (amplitude B/amplitude A) of the heart-lung field, it can be seen that the value of the average signal value ratio (amplitude B/amplitude A) of the heart-lung field tends to increase as the pulmonary valve regurgitation rate (%) increases (the regurgitation symptom becomes more severe).

As described above, a correspondence relationship is recognized between the average signal value ratio (amplitude B/amplitude A) of the heart-lung field and the pulmonary valve regurgitation rate (%). Therefore, the controller 31 generates information on the heart-lung field average signal value ratio (amplitude B/amplitude A) as “information on pulmonary valve regurgitation” from the dynamic image, and outputs and displays the information on the display part 34 or the like, thereby indicating the presence or absence of the pulmonary valve regurgitation and the degree of the regurgitation rate (level and severity of pulmonary valve regurgitation) when regurgitation is present to a user such as a doctor. Accordingly, the controller 31 of the analysis apparatus 3 can support the user's diagnosis of the pulmonary valve regurgitation. In this case, even if an ultrasonic echo examination or an MRI examination is not performed, it is possible to provide the user with information having the same accuracy as that of an echo examination or the like.

Furthermore, when the controller 31 further acquires parameters from the blood flow waveform obtained based on the signal values of the pixels, the controller 31 may set the threshold value using these parameters. The parameters are “skewness”, “kurtosis”, and the like. In a case where the controller 31 sets the threshold value, the “information on pulmonary valve regurgitation” generated from the dynamic image by the controller 31 may be a result of determining whether the patient to be diagnosed has values equal to or greater than the threshold value.

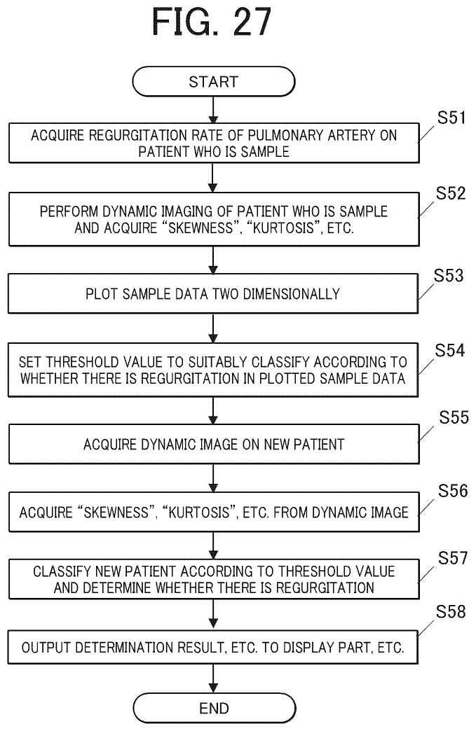

In this case, for example, the user first performs an examination with guaranteed reliability, such as an MRI examination, on the patient who is the sample, and as illustrated in FIG. 27, the controller 31 acquires the regurgitation rate of the pulmonary artery (step S51).

Then, the user performs dynamic imaging on the patient for whom the regurgitation rates have been acquired. The controller 31 obtains a blood flow waveform from the dynamic image and acquires values of items serving as parameters such as “skewness” and “kurtosis” (step S52). The controller 31 sets the data of the patient in which the regurgitation rate and the value serving as the parameter are acquired as sample data.

When the sample data acquired in this manner are accumulated, the controller 31 plots each piece of sample data two dimensionally by taking values of the parameters on the vertical axis and the horizontal axis, respectively (step S53). Then, the controller 31 sets a threshold value for each parameter such that the plotted sample data is correctly classified into the group of “with pulmonary valve regurgitation” and the group of “without pulmonary valve regurgitation” confirmed by the examination (step S54).

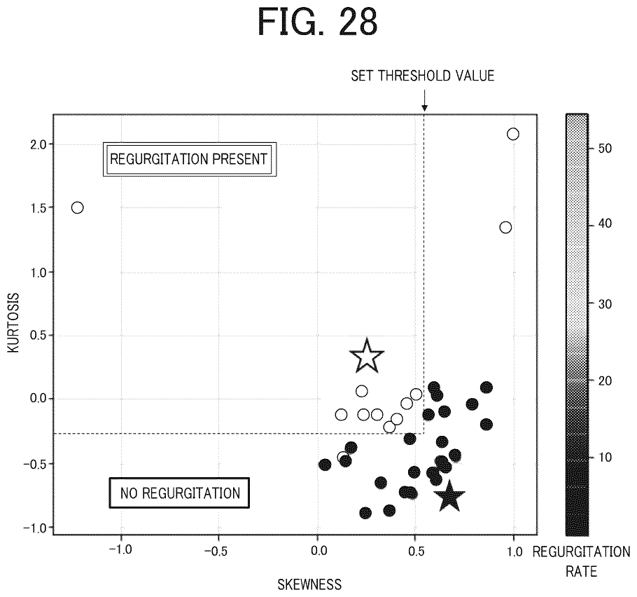

For example, FIG. 28 shows a result obtained by acquiring “skewness” and “kurtosis” as parameters and plotting sample data with “kurtosis” on the vertical axis and “skewness” on the horizontal axis. In FIG. 28, a white circle is a sample in which “pulmonary valve regurgitation is present” in an MRI examination or the like. In FIG. 28, the black circles are samples determined as “no pulmonary valve regurgitation”. As shown in FIG. 28, a patient with a high regurgitation rate tends to have a low “skewness” close to 0. In addition, a patient with a high regurgitation rate tends to have a high “kurtosis”. In addition, a patient having a high “skewness” or a not very high “kurtosis” tends to have a low regurgitation rate.

In the example shown in FIG. 28, the threshold value for the parameter “skewness” is set to 0.55 and the threshold value for the parameter “kurtosis” is set to −0.3 to classify the sample data. As a result, it was found that patients with a regurgitation rate of 25% or more can be classified into “pulmonary valve regurgitation present” with a classification accuracy of 94.3%.

In a case where the threshold values are set for the parameters (e.g., “skewness” and “kurtosis”) on the basis of the accumulation of the sample data in this way, when a patient who has not undergone an MRI examination or the like newly comes, first, a dynamic image is acquired (step S55). Then, the values of “skewness” and “kurtosis” are acquired by image analysis (step S56).

Then, the patients are classified according to threshold values such as “skewness” and “kurtosis”, and it is determined whether “there is pulmonary valve regurgitation” or “there is no pulmonary valve regurgitation” (step S57).

For example, in a case where the patient is classified into the position of the white star in FIG. 28, it is determined that “pulmonary valve regurgitation is present”. In a case where the patient is classified into the position of the black star in FIG. 28, it is determined that “there is no pulmonary valve regurgitation”.

The determination result of the classification is output from the controller 31 as an output unit to the display part 34 or the like (step S58) and is displayed.

The content output to the display part 34 or the like may be only the determination result. In addition, the result of plotting a new patient in the table as shown in FIG. 28 based on the values of “skewness” and “kurtosis” may be displayed on the display part 34 or the like.

The user such as the doctor sees the output result and checks the situation in more detail in a case where it is determined that there is pulmonary valve regurgitation in the patient, a case where the determined regurgitation rate is high, or the like. For this reason, the user takes measures such as giving an instruction to perform an MRI examination or the like.

When sample data is accumulated to the extent that it can be associated with the regurgitation rate, it is possible to derive not only “pulmonary valve regurgitation present” and “no pulmonary valve regurgitation” but also a more specific regurgitation rate (a quantitative value indicating the degree and/or severity of pulmonary valve regurgitation) from values such as “skewness” and “kurtosis”.

Note that by using multiple regression analysis or the like, the relation (correlation or the like) between parameters such as “skewness” and “kurtosis” and the pulmonary valve regurgitation rate may be considered, and the accuracy in determination of the regurgitation rate may be ensured.

According to the present embodiment, it is possible to perform blood flow analysis based on a dynamic image (X-ray dynamic image) that can be obtained relatively easily and non-invasively for a patient who is the subject M.

In the present embodiment, the processes of FIG. 3, FIG. 7, FIG. 15, and FIG. 27 are exemplified as the analysis process of the dynamic image. The analysis apparatus 3 may perform one or more of these processes or may perform all of these processes.

In a case where a plurality of kinds of “information on pulmonary valve regurgitation” are obtained by the controller 31 of the analysis apparatus 3 performing each process, all the information may be output to and displayed on the display part 34 or the like. Here, the user may also be able to select the information that the user desires to display. In addition, the controller 31 may generate more reliable information by multiplying various kinds of acquired information, output the final information, and provide the information to the user such as the doctor.

### [Effects]

As described above, the analysis apparatus 3, which is a dynamic image analysis apparatus according to the present embodiment, includes the controller 31 that acquires a dynamic image of the chest obtained by dynamic imaging using radiation, generates “information on pulmonary valve regurgitation” based on dynamic image information of “a site related to at least one of the pulmonary artery and the heart” in the dynamic image, and outputs the generated “information on pulmonary valve regurgitation”.

As described above, in the present embodiment, the controller 31 obtains “information on pulmonary valve regurgitation” using the dynamic image obtained by dynamic X-ray imaging. Therefore, for example, as compared with a conventional echo examination using an ultrasonic wave, an MRI examination, or the like, the examination can be performed relatively easily by using a general apparatus. In addition, it is only necessary to image a normal dynamic image. Therefore, there is also a lower risk that a variation occurs in a result obtained depending on the skill of an engineer as in, for example, an echo examination. It is non-invasive, X-ray exposure is relatively low, and the burden on the patient is also low. Furthermore, unlike MRI examination, there are no operational restrictions such as being able to be used for a patient whose body contains metal that is not compatible with MRI. Therefore, it is possible to widely cope with various needs.

Next, the controller 31 can obtain the “information on pulmonary valve regurgitation” with reliability equivalent to that of other examinations such as the ultrasonic echo examination and the MRI examination, by a method with less burden and restriction. Therefore, it is possible to support the diagnosis by the user such as the doctor.

Furthermore, in the present embodiment, the site (site related to the pulmonary artery) includes at least a region inside one of the right and left lung field regions and proximal portions of the right and left pulmonary arteries.

In the dynamic image, data is obtained from a region including right and left pulmonary artery proximal portions in which a blood flow state of the pulmonary artery necessary for determining pulmonary valve regurgitation can be viewed. Therefore, it is possible to obtain a signal value appropriate for determining the presence or absence of pulmonary valve regurgitation.

Furthermore, in the present embodiment, the site (site related to the pulmonary artery) is the target region that is inside the right and left lung field regions and is set in proximal parts of the right and left pulmonary arteries. The “information on pulmonary valve regurgitation” is the average value of the values in both the left and right target regions.

Therefore, an appropriate value can be obtained even for a patient in which the states of abnormalities such as pulmonary valve regurgitation differ between the right and left lungs. Thus, it is possible to support the diagnosis so that the correct diagnosis is performed.

Further, in the present embodiment, the dynamic image information is a signal value of a pixel. The controller 31 generates the “information on pulmonary valve regurgitation” based on the information obtained from the signal values of the pixels in the site (site related to the pulmonary artery).

In the dynamic image, data is obtained from a region including right and left pulmonary artery proximal portions in which a blood flow state of the pulmonary artery necessary for determining pulmonary valve regurgitation can be viewed. Therefore, it is possible to obtain a signal value appropriate for determining the presence or absence of pulmonary valve regurgitation.

Furthermore, the controller 31 may generate the “information on pulmonary valve regurgitation” based on the average value of the signal values of the pixels of the dynamic image. In this case, even when some of the signal values indicate peculiar values, the controller 31 can obtain appropriate information without being affected by the value.

According to the present embodiment, the “information on pulmonary valve regurgitation” is information on the presence or absence of the pulmonary valve regurgitation.

Thus, the user can know whether there is the pulmonary valve regurgitation from the dynamic image obtained by the X-ray dynamic imaging without performing the MRI examination or the like. Accordingly, it is possible to perform diagnosis support for easily diagnosing a pulmonary valve regurgitation patient or a patient suspected thereof.

Furthermore, it is possible to obtain information on the severity of pulmonary valve regurgitation, such as a regurgitation rate, from the dynamic image obtained by dynamic X-ray imaging. In this case, information similar to that obtained by the MRI examination can be obtained by a simple examination called X-ray dynamic imaging, and significant diagnosis support can be performed.

Further, the controller 31 can determine the presence or absence of the pulmonary valve regurgitation (or suspicion thereof) from the shape of the blood flow waveform. The controller 31 generates information related to the shape of the blood flow waveform and causes the display part 34 or the like to display the information, whereby it is possible to perform diagnosis support related to pulmonary valve regurgitation for the user such as the doctor.

Furthermore, the controller 31 may generate, as the “information on pulmonary valve regurgitation”, information visualizing the blood flow in the site (site related to the pulmonary artery). In this case, when the “information on pulmonary valve regurgitation” is displayed on the display part 34 or the like, it is easy to understand, and it is possible to appropriately support diagnosis.

In addition, the controller 31 may generate not the blood flow waveform itself but information (for example, “kurtosis”, “skewness”, “amplitude”, or the like) obtained on the basis of the blood flow waveform as “information on pulmonary valve regurgitation”. In this case, the controller 31 can also analyze pulmonary valve regurgitation multilaterally and quantitatively and can obtain significant information as a diagnostic support.

Further, the controller 31 may generate the “information on pulmonary valve regurgitation” on the basis of the index obtained at the part related to the pulmonary artery. In a case where the “information on pulmonary valve regurgitation” is generated as the “index” from, for example, the blood flow velocity, the blood flow volume amplitude), the ratio of the signal values of the heart and the lung field (amplitude ratio), and the like, it is possible to analyze the pulmonary valve regurgitation in multiple directions. Thus, it is possible to obtain information significant as a diagnostic support.

As described above, the controller 31 can extract various kinds of information from the dynamic image obtained by the X-ray dynamic imaging. Therefore, as illustrated in the present embodiment, the information can be used for diagnostic support for the user. Therefore, the analysis apparatus 3 can widely meet the needs of diagnostic support for pulmonary valve regurgitation, which has been difficult to diagnose only by limited methods such as ultrasonic echo examination and MRI examination.

### Modification Example

It is needless to say that the present invention is not limited to the above-described embodiments and can be appropriately modified without departing from the scope of the present invention.

For example, in the present embodiment, the case where the analysis apparatus 3 which is the dynamic image analysis apparatus includes the display part 34 which displays the dynamic image, the image indicating the analysis result of the image, and the presence or absence, the degree, and the like of the regurgitation has been exemplified. However, it is not essential that the analysis apparatus 3 includes the display part 34.

For example, a display device or the like including a high-resolution monitor or the like may be provided separately from the analysis apparatus 3 so that the analysis result or the like in the analysis apparatus 3 can be confirmed on the monitor such as the display device.

In addition, when a plurality of analysis results, such as an index indicating the presence or absence, the degree, or the like of a regurgitation and a determination result, are obtained, these analysis results may be displayed in parallel so as to be compared and examined.

Further, in a case where dynamic image imaging and various examinations are continuously and periodically performed on the same patient, information obtained therefrom may be stored and accumulated in the storage section 32 or the like. Next, in a case where a new examination or the like is performed, past data may also be displayed in parallel in a time-series manner so that newly obtained data and the past data can be compared with each other.

Thus, the doctor or the like can easily understand the progress of a symptom, the degree of improvement, and the like, and can provide effective information and support diagnosis in the case of performing continuous treatment.

Although some embodiments of the present invention have been described, the scope of the present invention is not limited to the above-described embodiments and includes the scope of the invention described in the claims and its equivalent scope.

Although embodiments of the present invention have been described and illustrated in detail, the disclosed embodiments are made for purposes of illustration and example only and not limitation. The scope of the present invention should be interpreted by terms of the appended claims.

What is claimed is:

1. A dynamic image analysis apparatus comprising: a hardware processor, wherein
the hardware processor is configured to,
execute processing to acquire a dynamic image of a chest obtained by dynamic imaging with radiation,
execute processing to generate information on pulmonary valve regurgitation based on dynamic image information of a site related to at least one of a pulmonary artery and a heart in the dynamic image, and
execute processing to output the generated information on pulmonary valve regurgitation.

2. The dynamic image analysis apparatus according to claim 1, wherein the site is at least an inside of any one of left and right lung field regions and includes a region of a proximal part of left and right pulmonary arteries.

3. The dynamic image analysis apparatus according to claim 1,
wherein,
the site is a target region set inside right and left lung field regions and in a proximal part of right and left pulmonary arteries, and
the information on pulmonary valve regurgitation is an average value of values in both left and right target regions.

4. The dynamic image analysis apparatus according to claim 1,
wherein,
the dynamic image information is a signal value of a pixel, and
the hardware processor generates the information on pulmonary valve regurgitation on the basis of information obtained from a signal value of a pixel in the site.

5. The dynamic image analysis apparatus according to claim 1,
wherein,
the dynamic image information is a signal value of a pixel, and
the hardware processor generates the information on pulmonary valve regurgitation on the basis of an average value of the signal value of the pixel in the site.

6. The dynamic image analysis apparatus according to claim 1, wherein the information on pulmonary valve regurgitation is information on presence or absence of pulmonary valve regurgitation.

7. The dynamic image analysis apparatus according to claim 1, wherein the information on pulmonary valve regurgitation is information on severity of pulmonary valve regurgitation.

8. The dynamic image analysis apparatus according to claim 1, wherein the hardware processor generates information on a shape of a blood flow waveform at the site as the information on pulmonary valve regurgitation.

9. The dynamic image analysis apparatus according to claim 1, wherein the hardware processor generates information in which a blood flow in the site is visualized as the information on pulmonary valve regurgitation.

10. The dynamic image analysis apparatus according to claim 1, wherein the hardware processor generates the information on pulmonary valve regurgitation on the basis of a blood flow waveform in the site.

11. The dynamic image analysis apparatus according to claim 1, wherein the hardware processor generates the information on pulmonary valve regurgitation based on an index obtained in the site.

12. A non-transitory computer-readable recording medium storing a program, wherein the program, when executed by a hardware processor, causes the hardware processor to perform operations comprising:
a process of acquiring a dynamic image of a chest obtained by dynamic imaging with radiation;
a process of generating information on pulmonary valve regurgitation based on dynamic image information of a site related to at least one of a pulmonary artery and a heart in the dynamic image; and
a process for outputting the generated information on pulmonary valve regurgitation.

13. A dynamic image analysis method comprising:
acquiring a dynamic image of a chest obtained by dynamic imaging with radiation;
generating information on pulmonary valve regurgitation based on dynamic image information of a site related to at least one of a pulmonary artery and a heart in the dynamic image; and
outputting the generated information on pulmonary valve regurgitation.
